(19) United States
(12) Patent Application Publication
(10) Pub. No.: US 2024/0108366 A1
(43) Pub. Date: Apr. 4, 2024

(54) SYSTEMS, APPARATUS AND METHODS FOR PROCESSING AUTOLOGOUS BLOOD FOR REINFUSION INTO A PATIENT

(71) Applicant: Retriever Medical, Inc., Irvine, CA (US)

(72) Inventors: Michael Bruce Horowitz, Naples, FL (US); Benjamin William Bobo, Las Vegas, NV (US); Brandon Matthew Repko, Mars, PA (US); Andreas Tzakis, Miami, FL (US)

(21) Appl. No.: 18/538,672

(22) Filed: Dec. 13, 2023

Related U.S. Application Data
(63) Continuation-in-part of application No. 18/220,373, filed on Jul. 11, 2023.
(60) Provisional application No. 63/368,325, filed on Jul. 13, 2022.

(51) Int. Cl. A61B 17/22; A61M 5/165

(52) U.S. Cl. CPC A61B 17/22; A61M 5/165; A61B 2217/005

(57) ABSTRACT

A method for processing autologous blood for reinfusion into a patient. The method includes the step of providing a blood reinfusion system adapted to isolate and extract impurities mixed with autologous blood aspirated from an incision site of the patient for reinfusion into the subject.

### CROSS-REFERENCES TO RELATED APPLICATIONS

The present application is a continuation-in-part of U.S. application Ser. No. 18/220,373, filed on Jul. 11, 2023, which claims the benefit of U.S. Pat. App. No. 63/368,325, filed on Jul. 13, 2022.

### FIELD OF THE INVENTION

The present invention relates generally to blood reinfusion systems and methods. More particularly, the present invention relates to systems, apparatus and methods for processing autologous blood for reinfusion into a patient.

### BACKGROUND OF THE INVENTION

As is well established, blood loss is an inevitable aspect of many invasive surgical procedures and, if not managed or accounted for, can lead to various significant adverse physiological conditions.

Indeed, a loss of over 20% of blood volume (˜900-1000 cc) during a surgical procedure can cause hypovolemic shock and a loss of over 50% of blood volume (˜2250-3500 cc) can cause cardiac arrest.

Blood loss during a surgical procedure can also result in post-procedure anemia, which can, and often will, hinder recovery.

Various means have thus been employed to manage blood loss during a surgical procedure. The most common means is transfusion of blood during and after the procedure.

As is well established, there are, however, several significant drawbacks and disadvantages associated with transfusion of blood to a patient during and after a surgical procedure.

A major problem associated with a typical blood transfusion, is that such blood is typically non-autologous (i.e., donated by another person), and thus, can induce various adverse physiological events, such as antigen reactions and disease transfer, if not properly screened.

Various blood recycling or reinfusion systems have thus been developed to address blood loss during a surgical procedure. Such systems include the Cell Saver® Elite®+ autotransfusion system developed by Haemonetics, the CATSmart® continuous autotransfusion system developed by Fresenius Kabi, the XTRA® autotransfusion system developed by LivaNova, and the autoLog® autotransfusion system developed by Medtronic.

The noted systems typically include means for collecting blood from a patient during a surgical procedure, means for processing the collected blood and means for reinfusing the blood into the patient.

As discussed below, there are similarly numerous drawbacks and disadvantages associated with the noted “blood reinfusion” systems.

A major disadvantage associated with the noted blood reinfusion systems is that the blood processing means of the systems can, and often will, damage the erythrocytes (i.e., red blood cells) in the collected “autologous” blood, which can, and often will, compromise the quality of the blood.

A further major disadvantage associated with the noted blood reinfusion systems is that the blood processing means typically includes mixing the collected “autologous” blood with a physiological solution (e.g., saline or Ringer's Solution) and centrifuging the mixed blood to isolate and recover the erythrocytes for reinfusion into the patient. The lighter portion of centrifuged mixed blood (i.e., the lighter plasma and buffy coat of the whole blood), which contains platelets, white blood cells, plasma proteins, and antibodies, are typically discarded as waste. The reinfused blood is thus devoid of the highly important platelets, white blood cells, plasma proteins, and antibodies.

A further disadvantage associated with the noted blood reinfusion systems is that such systems typically comprise large, complex equipment that is very difficult to operate and require multiple specialized technicians to operate. The systems thus often require advanced planning prior to use, including scheduling specialized technicians trained to set up and use the systems, and, hence, are also suboptimal for emergency use, e.g., instances of unexpected blood loss during a medical procedure or military combat.

A further disadvantage associated with the noted blood reinfusion systems is that they are typically not configured and/or adapted for use in sterile environments.

A further disadvantage associated with the noted blood reinfusion systems is the high costs associated with reinfusing blood into a patient therewith, i.e., reinfusion system acquisition and labor costs. As a result, such systems are typically not economically feasible for use during surgical procedures in developing countries.

It would thus be desirable to provide improved blood reinfusion systems that substantially reduce or eliminate the drawbacks and disadvantages associated with conventional blood reinfusion systems.

It is therefore an object of the present invention to provide improved blood reinfusion systems that substantially reduce or eliminate the drawbacks and disadvantages associated with conventional blood reinfusion systems.

It is another object of the present invention to provide improved blood reinfusion systems adapted to process autologous blood with minimal, if any, effect on the quality of the blood.

It is another object of the present invention to provide improved blood reinfusion systems adapted to process autologous blood without damaging the erythrocytes in the blood.

It is another object of the present invention to provide improved blood reinfusion systems adapted to process autologous blood with minimal blood component loss; specifically, platelet, white blood cell, plasma protein, and antibody, loss.

It is another object of the present invention to provide improved blood reinfusion systems configured and adapted for use in sterile environments.

It is another object of the present invention to provide improved blood reinfusion systems that are simple to use and can be easily operated manually by a single operator.

It is another object of the present invention to provide improved blood reinfusion systems that can be promptly employed in emergency situations.

It is another object of the present invention to provide improved blood reinfusion systems that can be readily employed in a multitude of surgical and interventional medical procedures.

### SUMMARY OF THE INVENTION

The present invention is directed to systems, apparatus and methods for processing autologous blood for reinfusion into a patient. In some embodiments of the invention, there are thus provided systems for processing autologous blood for reinfusion into a patient (referred to hereinafter as “blood reinfusion systems”).

In one preferred embodiment of the invention, the blood reinfusion systems comprise a suction canister, a blood filter assembly and a blood collection container,
the suction canister configured and adapted to receive autologous blood and impurities mixed therewith from an incision site of a patient, the suction canister comprising a first filter adapted to isolate and extract at least a first portion of the impurities from the autologous blood, whereby first processed autologous blood is obtained,
the blood filter assembly comprising a second filter adapted to receive the first processed autologous blood from the suction canister and isolate and extract at least a second portion of the impurities from the autologous blood, whereby second processed autologous blood is obtained, a third filter adapted to receive the second processed autologous blood from the second filter and isolate and extract at least a third portion of the impurities from the autologous blood, whereby third processed autologous blood is obtained, and a fourth filter adapted to receive the third processed autologous blood from the third filter and isolate and extract at least a fourth portion of the impurities from the autologous blood, whereby fourth processed autologous blood is obtained,
the blood collection container adapted to receive the fourth processed autologous blood from the blood filter assembly.

In some embodiments, the first filter comprises a pore size in the range of 1.0 mm to 5.0 mm.

In some embodiments, the second filter comprises a pore size in the range of 1.0 mm to 5.0 mm, the third filter comprises a pore size in the range of 40.0 micron to 1000.0 micron and the fourth filter comprises a pore size in a range of 10.0 micron to 40.0 micron.

In some embodiments of the invention, the suction canister further comprises sensor means adapted to monitor the volume of autologous blood contained in the suction canister.

In some embodiments, the blood filter assembly further comprises sensor means adapted to monitor flow of the autologous blood through the blood filter assembly.

In some embodiments, the blood filter assembly further comprises negative pressure means for providing negative pressure in the blood filter assembly, whereby the first processed autologous blood passes through the second filter, the second processed autologous blood passes through the third filter and the third processed autologous blood passes through the fourth filter via the negative pressure in the blood filter assembly.

In some embodiments of the invention, there are thus also provided methods for processing autologous blood for reinfusion into a patient. In one preferred embodiment of the invention, the methods comprise the steps of:
providing aspiration means for aspirating autologous blood and impurities mixed therewith from a patient, the aspiration means comprising an aspiration catheter configured and adapted to be positioned proximate an incision site of the patient;
providing a blood reinfusion system adapted to receive the mixture of the autologous blood and impurities from the aspiration means, the blood reinfusion system further adapted to isolate and extract the impurities mixed with the autologous blood,
the blood reinfusion system comprising a suction canister, a blood filter assembly and a blood collection container,
the suction canister being in fluid communication with the aspiration catheter of the aspiration means, the suction canister configured and adapted to directly receive the mixture of the autologous blood and impurities mixed therewith from the aspiration means,
the suction canister comprising a first filter comprising a pore size in the range of 1.0 mm to 5.0 mm, the first filter adapted to isolate and extract at least a first portion of the impurities mixed with the autologous blood, whereby first processed autologous blood is obtained,
the blood filter assembly comprising a first reservoir in fluid communication with the suction canister, the first reservoir configured and adapted to receive the first processed autologous blood from the suction canister,
the first reservoir comprising at least a second filter comprising a pore size in the range of 1.0 mm to 5.0 mm, the second filter adapted to isolate and extract at least a second portion of the impurities mixed with the autologous blood, whereby second processed autologous blood is obtained,
the blood filter assembly further comprising a third filter comprising a pore size in the range of 40.0 micron to 1000.0 micron, and a fourth filter comprising a pore size in a range of 10.0 micron to 40.0 micron,
the third filter being in fluid communication with the second filter and adapted to receive the second processed autologous blood from the second filter, the third filter adapted to isolate and extract at least a third portion of the impurities mixed with the autologous blood, whereby third processed autologous blood is obtained,
the fourth filter being in fluid communication with the third filter and adapted to receive the third processed autologous blood from the third filter, the fourth filter adapted to isolate and extract at least a fourth portion of the impurities mixed with the autologous blood, whereby fourth processed autologous blood is obtained,
the blood filter assembly further comprising a second reservoir in fluid communication with the fourth filter of the blood filter assembly, the second reservoir configured and adapted to receive the fourth processed autologous blood,
the blood collection container being in fluid communication with the blood filter assembly, the blood collection container configured and adapted to receive the fourth processed autologous blood from the second reservoir of the blood filter assembly;
coupling the aspiration catheter of the aspiration means to the suction canister of the blood reinfusion means;
positioning the aspiration catheter at a first incision site of the subject exhibiting autologous blood;
aspirating the autologous blood and impurities mixed therewith with the aspiration catheter, wherein the mixture of the autologous blood and the impurities is introduced into and through the aspiration catheter and into the suction catheter, into and through the blood filter assembly, and into the blood collection container, whereby the fourth processed autologous blood is contained therein.

In some embodiments, the first processed autologous blood passes through the second filter, the second processed autologous blood passes through the third filter and the third processed autologous blood passes through the fourth filter via a force of gravity.

In some embodiments, the blood filter assembly further comprises negative pressure means for providing negative pressure in the blood filter assembly, whereby the first processed autologous blood passes through the second filter, the second processed autologous blood passes through the third filter and the third processed autologous blood passes through the fourth filter via the negative pressure in the blood filter assembly.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 depicts a schematic illustration of one embodiment of a blood reinfusion system, in accordance with the invention;

FIG. 2 depicts a front plan view of the blood reinfusion system depicted in FIG. 1, in accordance with the invention;

FIG. 3A depicts a front plan view of one embodiment of a suction container, in accordance with the invention;

FIG. 3B depicts a top plan view of one embodiment of a suction container filter, in accordance with the invention;

FIG. 4 depicts a front plan view of one embodiment of a blood filter assembly, in accordance with the invention;

FIG. 5 depicts a schematic illustration of another embodiment of a blood reinfusion system, in accordance with the invention;

FIG. 6 depicts a schematic illustration of another embodiment of a blood reinfusion system, in accordance with the invention;

FIG. 7 depicts a front plan view of one embodiment of a blood collection container, in accordance with the invention;

FIG. 8 depicts a schematic illustration of another embodiment of a blood reinfusion system, in accordance with the invention;

FIG. 9 depicts a schematic illustration of another embodiment of a blood reinfusion system, in accordance with the invention;

FIG. 10 depicts a schematic illustration of another embodiment of a blood reinfusion system, in accordance with the invention;

FIG. 11 depicts a front plan view of the blood reinfusion system depicted in FIG. 10, in accordance with the invention;

FIG. 12 depicts a front plan view of one embodiment of a blood collection bag, in accordance with the invention;

FIG. 13 depicts a schematic illustration of another embodiment of a blood reinfusion system, in accordance with the invention;

FIG. 14 depicts a schematic illustration of another embodiment of a blood reinfusion system, in accordance with the invention;

FIG. 15 depicts a perspective view of one embodiment of a thrombectomy system, in accordance with the invention;

FIG. 16A depicts an illustration of thrombosed bovine blood, in accordance with the invention;

FIG. 16B depicts an illustration of blood impurities captured in a filter of a blood filter assembly, in accordance with the invention; and

FIG. 16C depicts an illustration of a 40 μm filter after a portion of blood processed in the blood filter assembly depicted in FIG. 4 is filtered therewith, in accordance with the invention.

### DETAILED DESCRIPTION OF THE PREFERRED EMBODIMENT

Before describing the present invention in detail, it is to be understood that this invention is not limited to particularly exemplified systems, apparatus, structures or methods as such may, of course, vary. Thus, although a number of systems, apparatus, structures and methods similar or equivalent to those described herein can be used in the practice of the present invention, the preferred systems, apparatus, structures and methods are described herein.

It is to be understood that the terminology used herein is for the purpose of describing particular embodiments of the invention only and is not intended to be limiting. The present invention is thus to be accorded the widest scope encompassing numerous alternatives, modifications and equivalents consistent with the principles and features disclosed.

It is also to be understood that language used in this specification should not be interpreted as a general disavowal of any one specific embodiment or used to limit the claims beyond the meaning of the terms used therein.

Further, unless defined otherwise, all technical and scientific terms used in this specification have the same meaning as commonly understood by one having ordinary skill in the art to which the invention pertains.

It is also understood that the general principles defined herein can be applied to other embodiments and applications without departing from the spirit and scope of the invention.

It is also understood that any feature or component described in association with a specific embodiment may be used and implemented with any other embodiment unless clearly indicated otherwise.

Further, all publications, patents and patent applications cited herein, whether supra or infra, are hereby incorporated by reference in their entirety.

### Definitions

The term “surgical procedure”, as used herein, means an invasive medical procedure characterized by purposeful/deliberate access to the body via an incision or percutaneous puncture, where blood can, and often will be exhibited.

The term “surgical procedure”, as used herein, thus includes, without limitation, the following surgical procedures: cardiac surgery procedures, such as coronary artery bypass grafting (CABG), valve replacement and repair, and aortic aneurysm repair; orthopedic surgery procedures; spinal surgery procedures; neurosurgery procedures, such as craniotomy; tumor resection procedures; organ transplant procedures; and trauma surgery procedures, such as trauma resuscitation and emergency surgical hemostasis.

The term “surgical procedure”, as used herein, also includes, without limitation, interventional cardiology procedures, such as coronary angiography, percutaneous coronary intervention (PCI), angioplasty, coronary stent placement, atherectomy, and transcatheter aortic valve replacement (TAVR); interventional vascular surgery procedures, such as endovascular aneurysm repair; interventional neurosurgery procedures, such as aneurysm coiling and arteriovenous malformation (AVM) procedures; and interventional trauma procedures.

The term “impurity”, as used herein in connection with autologous blood, means and includes blood clots, tissue debris, hair, foreign particles, activated coagulation factors, denatured proteins, plasma free hemoglobin, and any other fluid (e.g., irrigation fluid) introduced into the surgical site by medical personnel.

The terms “thrombus” and “occlusion” are used interchangeably herein and mean and include unwanted or undesired material disposed in a patient's veins or arteries that is partially or completely obstructing the flow of blood.

The terms “processed blood” and “purified blood” are also used interchangeably herein and mean autologous blood substantially devoid of impurities and unwanted cellular and blood components.

The terms “one embodiment”, “one aspect”, “an embodiment” and “an aspect”, as used herein, mean that a particular feature, structure, or characteristic described in connection with the embodiment can be included in at least one embodiment and not that any particular embodiment is required to have a particular feature, structure or characteristic described herein unless set forth in the claim.

The phrase “in one embodiment” or similar phrases employed herein do not limit the inclusion of a particular element of the invention to a single embodiment. The element can thus be included in other, or all embodiments discussed herein.

The term “substantially”, as used herein, means and includes the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof.

Use of the term “substantially” is equally applicable when used in a negative connotation to refer to the complete or near complete lack of an action, characteristic, property, state, structure, item, or result. For example, a structure which is “substantially free of” a bottom would either completely lack a bottom or so nearly completely lack a bottom that the effect would be effectively the same as if it completely lacked a bottom.

The term “comprise” and variations of the term, such as “comprising” and “comprises,” means “including, but not limited to” and is not intended to exclude, for example, other components, elements or steps.

The following disclosure is provided to further explain in an enabling fashion the best modes of performing one or more embodiments of the present invention. The disclosure is further offered to enhance the understanding and appreciation for the inventive principles and advantages thereof, rather than to limit in any manner the invention. The invention is defined solely by the appended claims, including any amendments made during the pendency of this application, and all equivalents of those claims as issued.

As indicated above, the present invention relates to blood reinfusion systems, apparatus and methods, i.e., systems, apparatus and methods adapted to process autologous blood for reinfusion into a patient. As discussed in detail herein, the blood reinfusion systems, apparatus and methods provide numerous significant advantages over conventional blood reinfusion systems. Among the advantages are the following:
means for processing autologous blood with minimal, if any, effect on the quality of the blood;
means for processing autologous blood without damaging the erythrocytes in the blood;
means for processing autologous blood with minimal blood component; specifically, platelet, white blood cell, plasma protein, and antibody, loss;
blood reinfusion systems, apparatus and methods that can be employed in sterile environments; and
blood reinfusion systems, apparatus and methods that are simple to use and can be easily operated manually by a single operator.

A further advantage of the blood reinfusion systems, apparatus and methods of the invention is that they can be promptly and readily employed during a multitude of surgical and interventional medical procedures, including, without limitation, invasive cardiac procedures, such as coronary artery bypass grafting (CABG), valve replacement and repair, and aortic aneurysm repair; orthopedic surgery procedures; spinal surgery procedures; neurosurgery procedures, such as craniotomy; tumor resection procedures; organ transplant procedures; thrombectomy procedures; interventional cardiology procedures, such as percutaneous coronary intervention (PCI) and transcatheter aortic valve replacement (TAVR); interventional vascular procedures, such as endovascular aneurysm repair; interventional neurosurgery procedures, such as aneurysm coiling and arteriovenous malformation (AVM) procedures; and various trauma procedures.

As indicated above, the blood reinfusion systems, apparatus and methods of the invention can also be readily employed at temporary trauma sites, such as a field hospital or trauma center in a combat zone, and permanent trauma treatment facilities and centers, such as in an emergency room or an intensive care unit (ICU).

As discussed in detail below, in a preferred embodiment, the blood reinfusion systems of the invention comprise (i) first blood collection means in communication with the aspiration means adapted to receive the aspirated autologous blood, (ii) blood processing means in communication with the first blood collection means adapted to process the collected autologous blood, and (iii) second blood collection means in communication with the processing means adapted to receive the autologous blood after processing.

As also discussed in detail herein, in some embodiments, the blood reinfusion systems of the invention comprise multiple separate first blood collection means and/or multiple separate blood collection means.

In some embodiments, the blood reinfusion systems of the invention comprise modular systems, i.e., the blood collection means is detachably coupled to the processing means.

In some embodiments, the blood reinfusion systems of the invention further comprise sensor means adapted to monitor blood volume in the first blood collection means and/or blood flow through the blood processing means.

In some embodiments, the blood reinfusion systems of the invention further comprise (i) aspiration means configured and adapted to collect autologous blood from a surgical site of a patient and (ii) control means programmed to control the aspiration means.

In some embodiments of the invention, the blood reinfusion systems further comprise integral means for reinfusing the processed (i.e., purified) autologous blood into the patient.

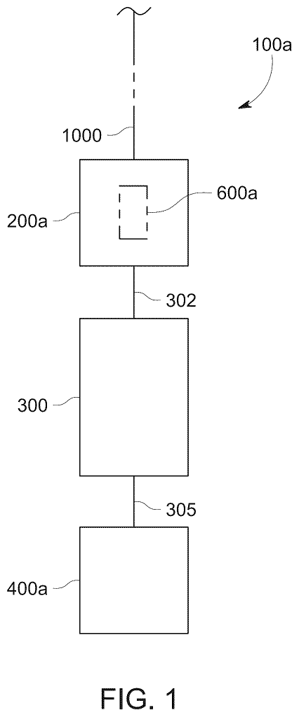

Referring now to FIG. 1, there is depicted a schematic illustration of one embodiment of a blood reinfusion system of the invention (denoted “100a”).

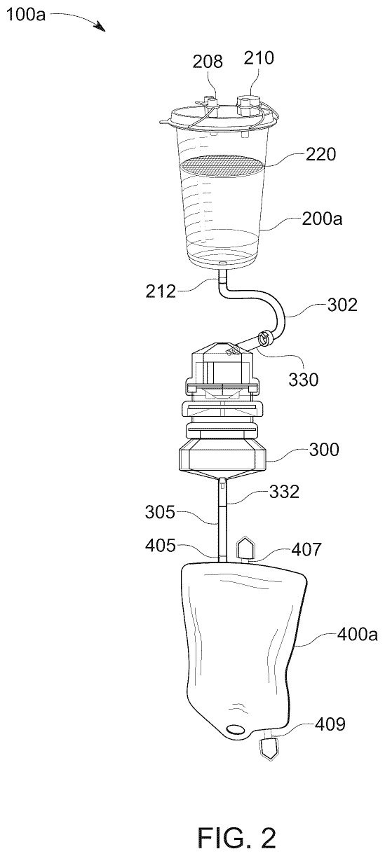

As depicted in FIGS. 1 and 2 and indicated above, in a preferred embodiment, the blood reinfusion system 100a comprises a stand-alone system, comprising first blood collection means (denoted “200a” and referred to herein as a “suction canister”), blood processing means (denoted “300” and referred to herein as a “blood filter assembly”), and second blood collection means (denoted “400a” and referred to herein as a “blood collection container”).

Each of the noted system components is described in detail below.

### Suction Canister

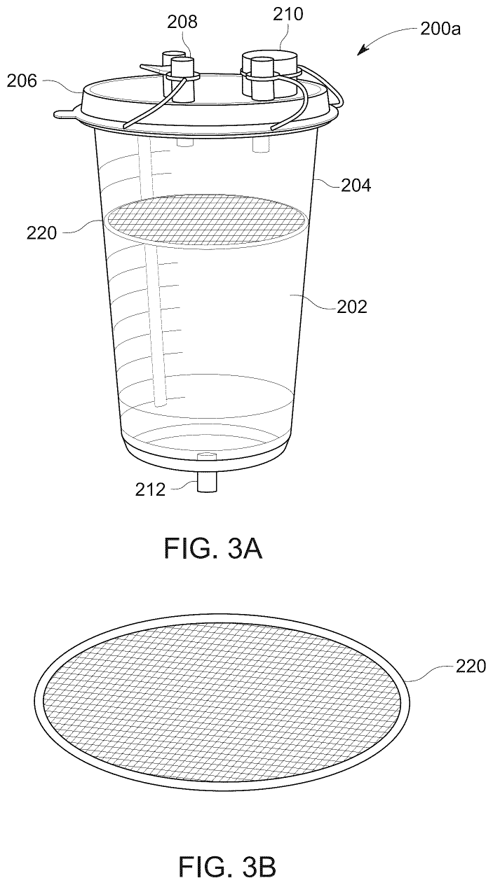

Referring now to FIGS. 2, 3A and 3B, there is depicted one embodiment of a suction canister 200a of the invention.

As depicted in FIG. 3A, in a preferred embodiment, the suction canister 200a comprises a primary fluid reservoir or housing 202 and a top cap 206, which, according to the invention and depicted in FIG. 3A, is sized and configured to sealably engage the top open portion 204 of the suction canister reservoir 202.

As further depicted in FIG. 3A, the suction canister cap 206 comprises a blood inlet 208, which is sized and configured to receive a conventional surgical aspiration catheter 1000 and, hence, blood transported therethrough; particularly, autologous blood and impurities mixed therewith aspirated from an incision site of a patient.

As additionally depicted in FIG. 3A, the suction canister reservoir 202 comprises a blood outlet 212, which, as discussed below, is sized and configured to receive the blood filter inlet line (i.e., conduit means) 302 to facilitate communication of the suction canister 200a with the blood filter assembly 300, and an internal filter 220 (see also FIG. 3B).

According to the invention, the internal filter 220 can comprise any suitable pore size. In a preferred embodiment, the internal filter 220 comprises a pore size in the range of approximately 1.0 mm to 5.0 mm.

According to the invention, the suction canister 200a can comprise additional filters. Thus, in some embodiments, the suction canister 200a comprises at least one additional filter comprising a pore size of 300.0 microns or smaller.

Thus, according to the invention, the suction canister 200a is configured and adapted to receive autologous blood mixed with impurities from a patient and isolate and capture at least a first portion of the impurities mixed with the autologous blood, whereby first processed autologous blood is obtained.

As depicted in FIG. 1, in some embodiments of the invention, the suction canister 200a further comprises first sensor means 600a adapted to monitor blood volume in the suction canister reservoir 202.

According to the invention, the suction canister 200a can comprise any configuration and size. In a preferred embodiment, the suction canister 200a is sized and configured to receive and contain in the range of 200.0 ml to 1000.0 ml of autologous blood. In some embodiments of the invention, the suction canister 200a is sized and configured to receive and contain approximately 500.0 ml of autologous blood.

As discussed below, in some embodiments of the invention, the blood reinfusion system 100a comprises two suction canisters; each canister being in communication with the blood filter 300, discussed below.

### Blood Filter Assembly

As depicted in FIGS. 1 and 2, the blood filter assemblies of the invention are in communication with the suction canisters of the invention and are adapted to further process the autologous blood collected from a patient.

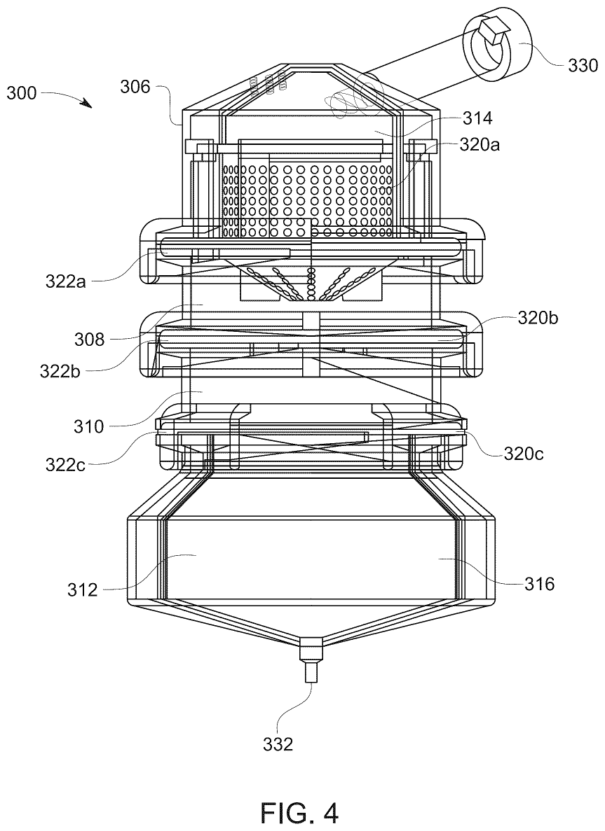

Referring now to FIGS. 2 and 4, one preferred embodiment of a blood filter assembly of the invention will be described in detail.

In a preferred embodiment, the blood filter assembly 300 comprises one embodiment of the filter assembly disclosed in priority U.S. application Ser. No. 18/220,373, which, as depicted in FIG. 4, comprises a three-stage filter system comprising a top housing portion 306, a first intermediate housing portion 308, a second intermediate housing portion 310, and a bottom housing portion 312; the top housing portion 306, first intermediate housing portion 308 and second intermediate housing portion 310 each comprising at least one filter.

According to the invention, the blood filter assembly 300 can also comprise a two-stage filter system comprising the top housing portion 306, first intermediate housing portion 308 and a bottom housing portion 312; the top housing portion 306 and first intermediate housing portion 308 similarly comprising at least one filter.

The blood filter assembly 300 can also comprise additional intermediate housing portions, such as a third and fourth intermediate housing portion, wherein each additional intermediate housing portion would similarly comprise at least one filter.

As set forth in priority U.S. application Ser. No. 18/220,373 and depicted in FIG. 4, the top housing portion 306 comprises a first reservoir 314 that is adapted and configured to receive collected autologous blood, in this instance, the first processed autologous blood transmitted from the suction canister 200a(i.e., autologous blood with at least a first portion of impurities mixed therewith extracted), and the bottom housing portion 312 comprises a second reservoir 316 that is adapted and configured to receive processed autologous blood.

In some embodiments, the first reservoir 314 preferably comprises a volume in a range of approximately 60.0 ml to 300.0 ml and the second reservoir 316 preferably comprises a volume in a range of approximately 100.0 ml to 400.0 ml.

As set forth in priority U.S. application Ser. No. 18/220,373, the top housing portion 306 of the blood filter assembly 300 comprises a first filter 320a, which preferably comprises a pore size in the range of approximately 1.0 mm to 5.0 mm, more preferably, a pore size of approximately 2.0 mm, the first intermediate housing portion 308 comprises a second filter 320b, which preferably comprises a pore size in the range of approximately 40.0 micron to 1000.0 micron (1.0 mm), more preferably, a pore size of approximately 250.0 micron, the second intermediate housing portion 310 comprises a third filter 320c, which preferably comprises a pore size in a range of approximately 10.0 micron to 40.0 micron, more preferably, a pore size of approximately 40.0 micron.

In a preferred embodiment of the invention, the first filter 320a is configured and adapted to receive the first processed autologous blood from the suction canister and isolate and extract first excess, i.e. remaining, impurities from the first processed autologous blood, whereby second processed autologous blood is obtained, the second filter 320b is adapted to receive the second processed autologous blood from the first filter 320a and isolate and extract second excess impurities from the second processed autologous blood, whereby third processed autologous blood is obtained, and the third filter 320c is adapted to receive the third processed autologous blood from the second filter 320b and isolate and extract third excess impurities from the second processed autologous blood, whereby purified autologous blood substantially devoid of blood clots, emboli, tissue debris, foreign particles, etc., is obtained.

According to the invention, the first, second and third filter 320a, 320b, 320c can comprise any acceptable surgical material, e.g., stainless steel, and form. In a preferred embodiment, the first filter 320a comprises a perforated filter and the second and third filters 320b, 320c comprise mesh filters.

In some embodiments of the invention, the first intermediate portion 308 or the second intermediate portion 310 of the blood filter 300 comprises or includes a membrane filter, comprising a pore size in the range of approximately 0.0001 micron to 100 micron.

In some embodiments of the invention, the second intermediate portion 310 of the blood filter 300 comprises or includes an emboli filter adapted to remove residual air, if any, from the processed autologous blood.

In a preferred embodiment, the top housing portion 306, first intermediate housing portion 308, and second intermediate housing portion 310 are detachably coupled in succession, whereby the autologous blood is successively filtered through filters 320a, 320b, and 320c via gravity.

In some embodiments of the invention, as discussed below, the blood filter assembly 300 includes means for providing negative pressure therein, wherein the autologous blood is successively filtered and, hence, processed through filters 320a, 320b, and 320c via the negative pressure in the blood filter assembly 300.

In a preferred embodiment, the top housing portion 306, the first intermediate housing portion 308, the second intermediate housing portion 310, and the bottom housing portion 312 are detachable from one another for ease of access and cleaning of the respective housing portions and cleaning and replacing the filters 320a, 320b, and 320c.

As set forth in priority U.S. application Ser. No. 18/220,373 and depicted in FIG. 4, the top housing portion 306 of the blood filter assembly 300 comprises an inlet port 330 adapted to receive the blood filter inlet line 302 and, hence, first processed autologous blood from the suction canister 200a transmitted therethrough.

In a preferred embodiment, the inlet port 330 comprises a luer connector to facilitate releasable connection of the suction canister 200a to the blood filter assembly 300.

As depicted in FIG. 4, the bottom housing portion 312 of the blood filter assembly 300 further comprises an outlet port 332, which is adapted to receive the blood filter outlet line 305 to facilitate transfer of the blood processed by the blood filter assembly 300, i.e., fourth processed autologous blood, to the blood collection container 400a.

In a preferred embodiment, the outlet port 332 similarly comprises a luer connector to facilitate releasable connection of the blood filter assembly 300 to the blood collection container 400a.

As further set forth in priority U.S. application Ser. No. 18/220,373, in some embodiments, the inner walls of the top housing portion 306 comprise channels that allow for the first filter 320a, when inserted into the top housing portion 306 from the bottom of the top housing portion 306 (when the top housing portion 306 is detached from the first intermediate housing portion 308), to be twisted in a first direction and be locked in place, and twisted in a second direction (opposite to the first direction) to unlock.

As also set forth in priority U.S. application Ser. No. 18/220,373, in some embodiments, a flow redirector element is positioned above and proximate each of the filters 320a, 320b, 320c, to bias and control the blood flow thereto, e.g., blood flow towards a side or portion of the filters. By configuring the flow redirector element in such manner, impurity accumulation is focused to a portion of the filters while the remaining portion(s) of the filters remains open and unobstructed.

According to the invention, the plane of the flow redirector element can be inclined at any desired predefined angle, e.g., 30.0 degrees to 45.0 degrees from a horizontal plane.

In some embodiments, the predefined angle of the flow redirector element ranges from approximately 0.0 degrees to 60.0 degrees from the horizontal plane.

As further set forth in priority U.S. application Ser. No. 18/220,373 and depicted in FIG. 4, the top housing portion 306, first intermediate housing portion 308, second intermediate housing portion 310, and bottom housing portion 312 of the blood filter assembly 300 are sealed, when connected, via a plurality of gaskets 322a, 322b, 322c and O-rings.

Further features and embodiments of the blood filter assembly 300 are set forth in U.S. application Ser. No. 18/220,373, which is expressly incorporated by reference herein in its entirety.

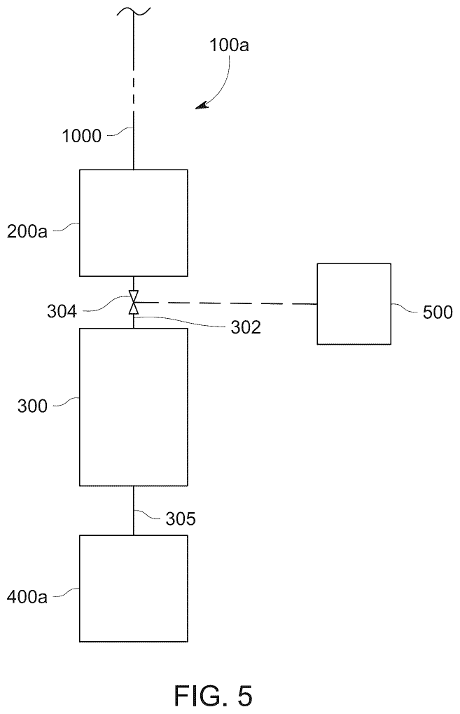

Referring now to FIG. 5, in some embodiments of the invention, the blood filter inlet line (i.e., conduit means) 302 comprises a valve assembly 304, which is adapted to modulate blood flow from the suction canister 200a into the blood filter assembly 300.

According to the invention, the valve assembly 304 can comprise any suitable valve assembly including, without limitation, a passive (one-way) valve assembly, an active valve assembly and a multi-way valve assembly.

As depicted in FIG. 5, in the noted embodiments, the blood reinfusion system 100a further comprises control means 500, which is programmed to control the valve assembly 304 and, hence, blood flow into the blood filter assembly 300.

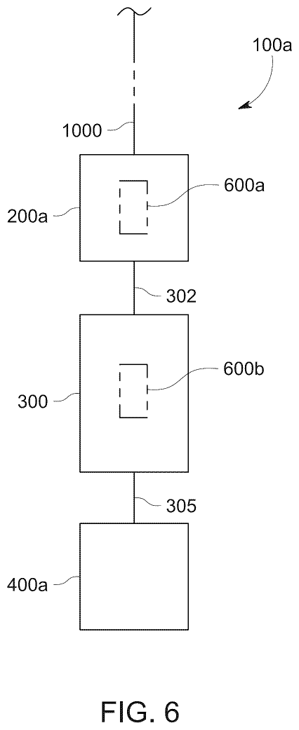

Referring now to FIG. 6, in some embodiments of the invention, the blood filter assembly 300 similarly further comprises a sensor system 600b, which is adapted to monitor blood flow through the blood filter assembly 300.

In some embodiments of the invention, the top housing potion 306 of the blood filter 300 further comprises an agent inlet configured and adapted to deliver blood processing agents and compositions into blood filter assembly 300, when it is desired to mix such agents and/or compositions with the autologous blood. In some embodiments, the blood processing agents and compositions are pre-loaded in the top housing portion 306 and/or bottom housing portion 312 in a powdered or lyophilized form.

Exemplar agents and compositions include, without limitation, anticoagulants, such as heparin or coumadin; thrombolytics, such as tissue plasminogen activator (tPA), streptokinase, or urokinase; and hormones, such as erythropoietin (EPO).

### Blood Collection Container(s)

As indicated above, the blood collection containers of the invention are configured and adapted to receive and contain the processed autologous blood from the blood filter assemblies of the invention.

According to the invention, the blood collection containers can comprise any configuration and size. In one preferred embodiment, the blood collection containers comprise a blood collection bag, such as a blood transfusion bag, to facilitate reinfusion of the processed autologous blood into a patient.

In the noted preferred embodiment, the blood collection bag preferably comprises a size or capacity in the range of 200 ml to 1000 ml.

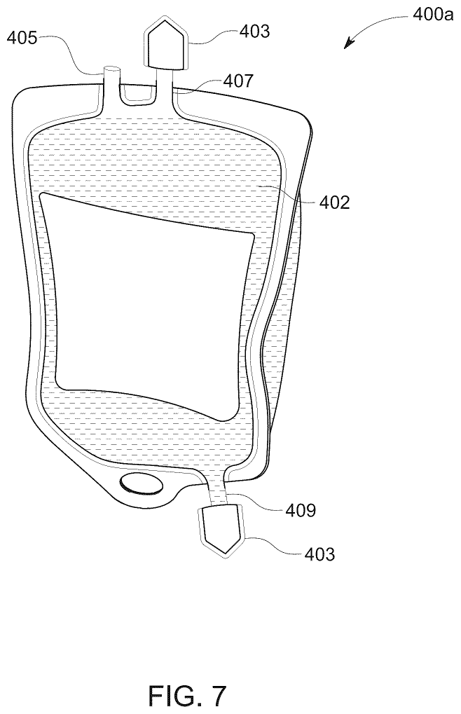

Referring now to FIG. 7, there is depicted one embodiment of a blood collection container of the invention in the form of a blood collection bag.

As depicted in FIG. 7, the blood collection container, i.e., bag, 400a comprises a sealed pouch comprising a blood inlet 405, which is sized and adapted to receive the blood filter outlet line 305 of the blood filter assembly 300 (see FIG. 2) and, hence, processed autologous blood (denoted “402”) from the blood filter assembly 300, an air vent 407, and a blood outlet 409 that is sized and adapted to receive a blood transfusion line to reinfuse the processed autologous blood into the patient.

As further depicted in FIG. 7, the air vent 407 and blood outlet 409 are further adapted to receive end caps 403, which are sized and adapted to close and seal the air vent 407 and blood outlet 409 when appropriate.

In some embodiments, one or more of the aforementioned blood processing agents and compositions are pre-loaded in the blood collection container 400a in a powdered or lyophilized form.

As indicated above, the blood reinfusion system 100a can further comprise two (2) blood collection containers; each adapted to couple to the blood filter assembly 300.

As indicated above, according to the invention, the blood reinfusion system 100a can also comprise a modular system, wherein suction canister 200a and blood filter assembly 300 are detachably coupled and thus the blood filter inlet line 302 of the blood filter assembly 300 is eliminated, or the suction canister 200a, blood filter assembly 300 and blood collection container 400a are detachably coupled and thus the blood filter inlet line 302 and blood filter outlet line 305 of the blood filter assembly 300 are eliminated.

According to the invention, the noted modular systems can further comprise on-off switches at the interconnections between the suction canister 200a and blood filter assembly 300, and the blood filter assembly 300 and bag(s) 400a, if part of the modular system.

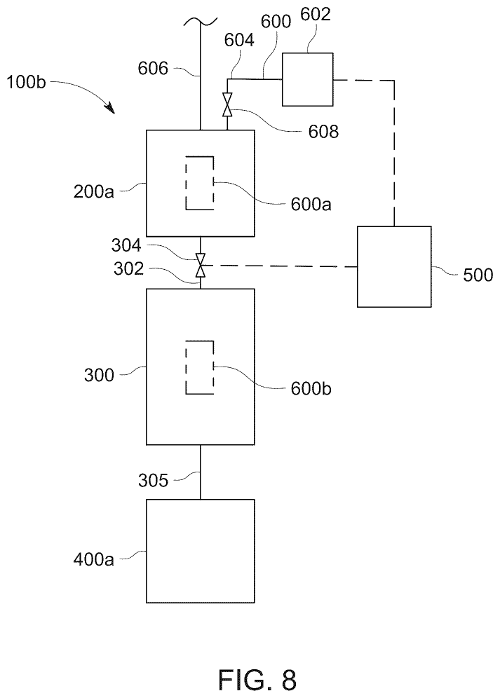

Referring now to FIG. 8, there is depicted a schematic illustration of a further embodiment of a blood reinfusion system of the invention (denoted “100b”).

As depicted in FIG. 8, the blood reinfusion system 100b similarly comprises suction canister 200a, blood filter assembly 300, and blood collection container 400a, discussed above. The blood reinfusion system 100b further comprises the control means 500.

As further depicted in FIG. 8, the blood reinfusion system 100b further comprises aspiration means 600, comprising a negative pressure (or suction) line 604, which is sized and configured to engage and, hence, communicate with the suction inlet 210 of the suction canister 200a, an aspiration catheter 606 adapted to be positioned proximate a surgical site of a patient, means for providing negative pressure and, hence, a suction force, though the aspiration catheter 606, and control means 500 for controlling the negative pressure means.

In a preferred embodiment, the negative pressure means, i.e., means for providing the suction force though the aspiration catheter 606, comprises a conventional pump assembly 602.

In a preferred embodiment, the pump assembly 602 is configured and adapted to generate and provide a negative pressure in the suction canister 200a via negative pressure line 604, which provides the suction force though the aspiration catheter 606 connected thereto.

In a preferred embodiment, the pump assembly 602 is configured and adapted to provide a negative pressure up to −400 mm Hg.

As further depicted in FIG. 8, in some embodiments, the aspiration means 600 further comprises a valve assembly 608, which is disposed in the negative pressure line 604. In the noted embodiments, the valve assembly 608 is adapted to modulate the negative pressure transmitted to the suction canister 200a and, hence, is also in communication with the control means 500 of the system 100b, which is additionally programmed to control the valve assembly 608 and, hence, negative pressure transmitted to the suction canister 200a.

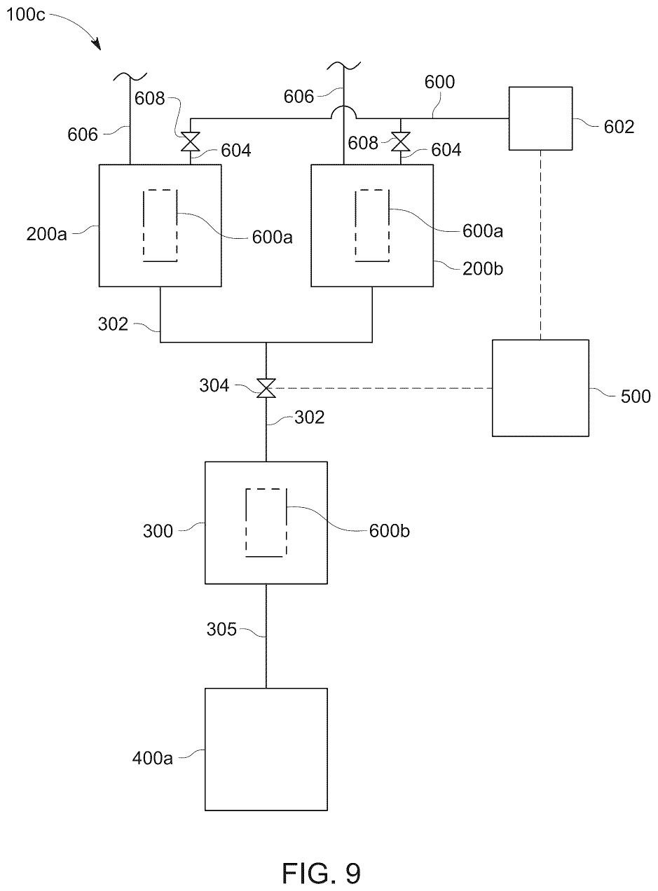

Referring now to FIG. 9, there is depicted a schematic illustration of a further embodiment of a blood reinfusion system of the invention (denoted “100c”).

As depicted in FIG. 9, the blood reinfusion system 100c similarly comprises the suction canister 200a, blood filter assembly 300, blood collection container 400a, control means 500 and aspiration means 600 depicted in FIG. 8 discussed above.

However, as depicted in FIG. 9, the blood reinfusion system 100c further comprises a second suction canister (denoted “200b”), which, according to the invention, is substantially similar in construction and function as suction canister 200a described above.

As depicted in FIG. 9, the second suction canister 200b is similarly in communication with the aspiration means 600 via negative pressure line 604, as described above, and blood filter assembly 300 via blood inlet line 302.

According to the invention, blood reinfusion systems 100b and 100c can also comprise modular systems, such as the modular blood reinfusion system 100a, described above.

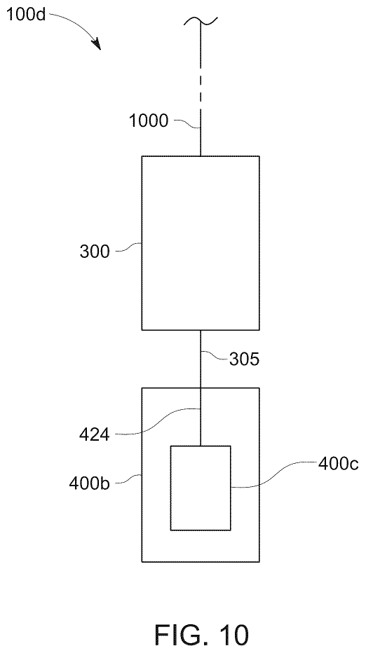

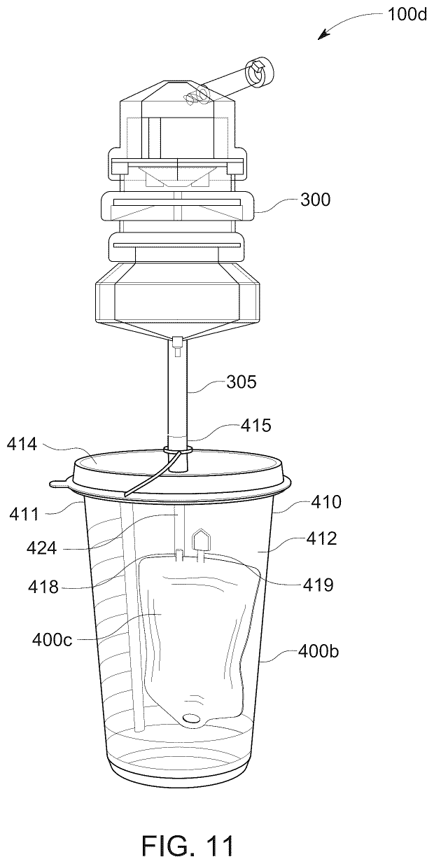

Referring now to FIGS. 10 and 11, there is depicted a schematic illustration of a further embodiment of a blood reinfusion system of the invention (denoted “100d”).

As depicted in FIGS. 10 and 11, the blood reinfusion system 100d similarly comprises the three-stage blood filter 300, discussed above. The blood reinfusion system 100d further comprises a unique blood collection container 400b.

As depicted in FIG. 11, the blood collection container 400b comprises an outer container 410, comprising an inner fluid reservoir 412 and a top cap 414, which, according to the invention, is similarly sized and configured to sealably engage the top open portion 411 of the outer container 410.

As further depicted in FIG. 11, the blood collection container 400b further comprises an inner blood collection container or bag 400c, which is disposed in the inner fluid reservoir 412 of the outer container 410.

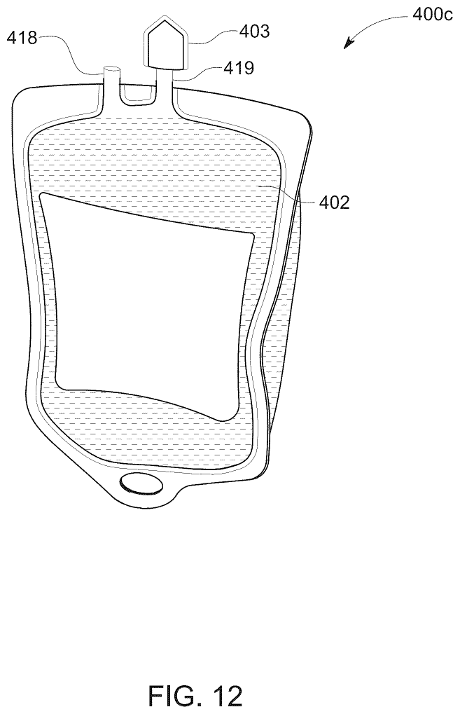

As additionally depicted in FIGS. 11 and 12, the inner blood collection bag 400c similarly comprises a sealed pouch comprising a blood inlet 418, which is sized and configured to receive the blood inlet line 424 of the bag 400c, and an air vent or filter 419, which is similarly sized and adapted to receive an end cap 403 when appropriate.

To facilitate communication of the inner blood collection bag 400c with the blood filter 300 and, hence, receipt of processed blood therefrom (denoted “402” in FIG. 12), in a preferred embodiment, the top cap 414 of the blood collection container 400b comprises a blood inlet 415, which is sized and configured to receive the blood filter outlet line 305 and the blood inlet line 424 of the inner blood collection bag 400c.

To facilitate the noted communication of the blood inlet 415 of the container cap 414 with the blood inlet line 424 of the inner blood collection bag 400c, the blood inlet 415 preferably extends into the inner fluid reservoir 412 of the outer container 410 when the top cap 414 is engaged thereto.

According to the invention, blood flow into and through the blood filter 300 and, thereby into the inner blood collection bag 400c is facilitated by the negative pressure (or vacuum) of the external aspiration catheter 1000 (and, hence, aspiration system). In addition to the processed blood transmitted through the blood filter 300, the inner blood collection bag 400c thus may, and, in all likelihood will, contain undesirable air. However, according to the invention, when the external aspiration catheter 1000 is disconnected from the blood filter 300 (and, hence, the negative pressure in the system 100d is released), the inner blood collection bag 400c relaxes and, hence, contracts, and the air in the bag 400c is released via air vent 419 when unsealed.

In a preferred embodiment, the outer container 410 of the blood collection container 400b comprises a rigid structure, such as, by way of example, a polypropylene housing or case, which secures the inner blood collection bag 400c in a sealed, sterile protective structure.

According to the invention, the blood filter outlet line 305 of the blood filter 300 can similarly comprise a valve assembly, such as valve assembly 304 depicted in FIG. 9, to modulate blood flow into the blood collection container 400b.

According to the invention, one or more of the aforementioned agents and compositions, e.g., anticoagulants, can be pre-loaded into the blood collection bag 400c in a powdered or lyophilized form.

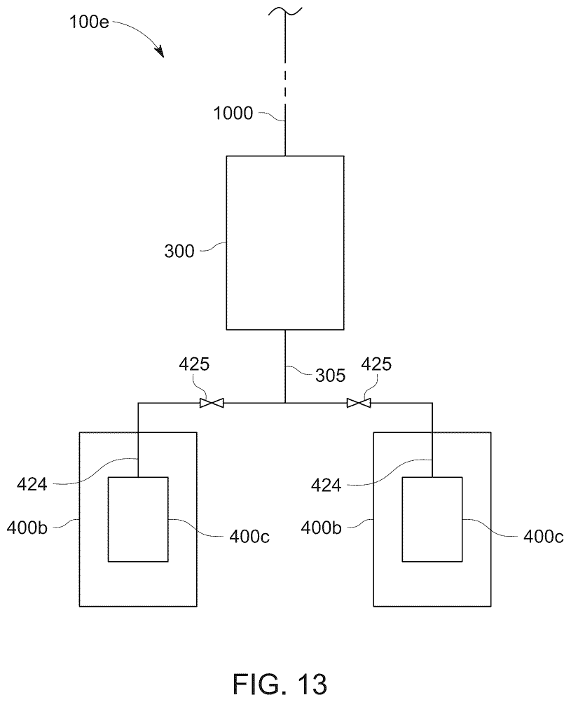

Referring now to FIG. 13, there is depicted a schematic illustration of a further embodiment of a blood reinfusion system of the invention (denoted “100e”).

As depicted in FIG. 13, the blood reinfusion system 100e is similar to blood reinfusion system 100d depicted in FIG. 10 and discussed above, except, in this embodiment, the blood reinfusion system 100e comprises two blood collection containers 400b.

As further depicted in FIG. 13, each blood collection container 400b is in fluid communication with the blood filter 300 via blood filter outlet line 305.

According to the invention, valve assemblies 425 can be disposed in the blood filter outlet line 305 proximate each blood collection container 400b to modulate blood flow into the containers 400b. In such embodiments, the blood reinfusion system 100e would further comprise control means programmed and configured to control the valve assemblies, such as control means 500 depicted in FIG. 5 and described above.

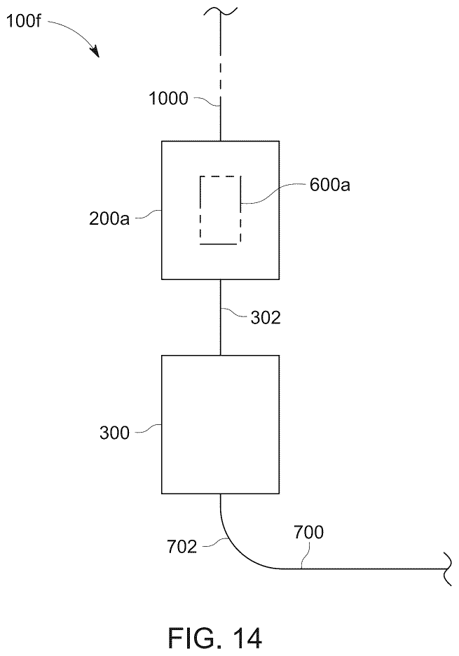

Referring now to FIG. 14, there is depicted a schematic illustration of a further embodiment of a blood reinfusion system of the invention (denoted “100f”).

As depicted in FIG. 14, the blood reinfusion system 100f similarly comprises the suction canister 200a and blood filter 300 of the base blood reinfusion system 100a depicted in FIGS. 1 and 2.

However, as further depicted in FIG. 14, the blood reinfusion system 100f further comprises patient blood infusing means (denoted “700”) adapted and configured to continuously reinfuse the processed and, hence, purified autologous blood into a patient during processing via the blood filter 300 of the system 100f.

According to the invention, the purified autologous blood is reinfused into the patient via a transfusion line (i.e., conduit means) 702, which is connected directly to the blood filter outlet line 305 of the blood filter 300.

According to the invention, the blood reinfusion system 100d can also comprise a modular system, wherein the blood filter assembly 300 and blood collection container 400b are interconnected and thus the blood filter outlet line 305 of the blood filter assembly 300 is eliminated.

According to the invention, the noted modular systems can similarly further comprise an on-off switch at the interconnection between the blood filter assembly 300 and blood collection container 400b.

According to the invention, the suction canisters 200a, 200b of the blood reinfusion systems of the invention can further comprise integral suction canister/filter assemblies, i.e., the suction canisters comprise an internal filter system comprising a plurality of filters.

According to the invention, the plurality of filters can comprise any suitable pore size, including, without limitation, the filter pore sizes referenced above.

Thus, in some embodiments of the invention, the internal filter system comprises three (3) separate filters: a first filter comprising a pore size in the range of approximately 1.0 mm to 5.0 mm, more preferably, a pore size of approximately 2.0 mm, a second filter comprising a pore size in the range of approximately 40.0 micron to 1000.0 micron (1.0 mm), more preferably, a pore size of approximately 250.0 micron, and a third filter comprising a pore size in a range of approximately 10.0 micron to 40.0 micron, more preferably, a pore size of approximately 40.0 micron.

In some embodiments of the invention, the internal filter system comprises four (4) separate filters: a first filter comprising a pore size in the range of approximately 3.0 mm to 5.0 mm, more preferably, a pore size of approximately 4.0 mm, a second filter comprising a pore size in the range of approximately 1.0 mm to 3.0 mm, more preferably, a pore size of approximately 2.0 mm, a third filter comprising a pore size in the range of approximately 40.0 micron to 1000.0 micron (1.0 mm), more preferably, a pore size of approximately 250.0 micron, and a fourth filter comprising a pore size in a range of approximately 10.0 micron to 40.0 micron, more preferably, a pore size of approximately 40.0 micron.

As indicated above, a major advantage of the blood reinfusion systems, apparatus and methods of the invention is that they can be promptly and readily employed during a multitude of surgical and interventional medical procedures, including, without limitation, invasive cardiac procedures, such as coronary artery bypass grafting (CABG), valve replacement and repair, and aortic aneurysm repair; orthopedic surgery procedures; spinal surgery procedures; neurosurgery procedures, such as craniotomy; tumor resection procedures; organ transplant procedures; thrombectomy procedures; interventional cardiology procedures, such as percutaneous coronary intervention (PCI) and transcatheter aortic valve replacement (TAVR); interventional vascular procedures, such as endovascular aneurysm repair; interventional neurosurgery procedures, such as aneurysm coiling and arteriovenous malformation (AVM) procedures; and various trauma procedures.

Exemplar procedures using a blood reinfusion system of the invention are set forth below.

### Operating Room (OR) Procedures

### Stabilization of a Dysfunctional Sacroiliac (SI) Joint

A SI joint prosthesis, such as prosthesis 70, depicted and described in U.S. application Ser. No. 18/107,563 is provided.

The OR aspiration system is initially engaged to at least one suction canister 200a, 200b (or both suction canisters 200a and 200b) of blood reinfusion system 100a, as depicted in FIG. 1.

An incision in and through tissue of the patient is made to provide posterior access to the patient's dysfunctional SI joint; preferably, a 2.0 cm to 3.0 cm incision.

The aspiration catheter, e.g., aspiration catheter 1000, is disposed proximate the incision site, i.e., body cavity formed via the incision, and engaged.

Thereafter, a guide bore is created in the dysfunctional SI joint, and a guide pin is inserted therein.

After the guide pin is inserted in the dysfunctional SI joint, a pilot opening is created in the dysfunctional SI joint with a tool assembly, the pilot opening comprising a first portion in the ilium bone structure and a second portion in the sacrum bone structure.

Thereafter, the tool assembly is removed, and the dysfunctional SI joint is flushed with a saline solution—the aspiration catheter 1000 continually aspirating the autologous blood of the patient, bone fragments, saline, etc. at the incision site and delivering the mixture of blood and other impurities into and through the blood reinfusion system 100a for processing by the blood filter assembly 300.

After the tool assembly is removed and the SI joint is flushed, the prosthesis 70 is advanced into the pilot opening in the SI joint.

After the procedure is completed and before the incision is sutured, the aspiration catheter 1000 is removed from the incision site and the aspiration system is disengaged.

The incision site is thereafter sutured and, hence, closed.

After the incision site is closed, the blood collection container 400a is disconnected from the blood filter assembly 300. A transfusion line is thereafter attached to the outlet 409 of the blood collection container 400a and the blood collection container 400a is mounted on an IV stand. Thereafter, the transfusion line is disposed in a blood vessel of the subject, wherein the processed autologous blood is reinfused into the patient.

### Thrombectomy Procedures

As indicated above, the blood reinfusion systems, apparatus and methods of the invention can also be readily employed during thrombectomy procedures to remove occlusions and unwanted matter, such as thrombi or clots, from an artery or vein in a patient.

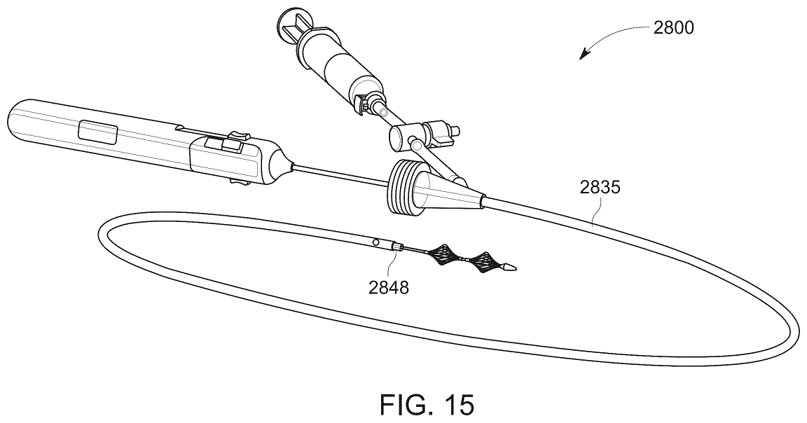

An exemplar thrombectomy procedure with the thrombectomy apparatus 2800 depicted in FIG. 15 (originally depicted in FIG. 28A of priority U.S. application Ser. No. 18/220,373 and referred to therein as a “retrieval apparatus”) is described below.

The OR aspiration system is initially connected to the aspiration catheter 2835 of the thrombectomy apparatus 2800. The aspiration catheter 2835 is thereafter connected to a blood reinfusion system of the invention, in this instance blood reinfusion system 100a.

The delivery catheter 2848 (with the aspiration catheter 2835 disposed therein, as described in priority U.S. application Ser. No. 18/220,373) is disposed in the patient's vessel, e.g., artery, proximate the occlusion, as described in priority U.S. application Ser. No. 18/220,373.

After the delivery catheter 2848 is disposed in the patient's vessel proximate the occlusion, the occlusion (and material thereof) is dislodged from the vessel with the thrombectomy apparatus 2800 and the occlusion (and material thereof) and autologous blood proximate thereto are aspirated into the aspiration catheter 2835, as described in U.S. application Ser. No. 18/220,373, and thereafter into the blood filter 300, wherein the autologous blood is processed, in this instance, the occlusion (and material thereof) is filtered from the autologous blood.

After the occlusion (and material thereof) is dislodged from the vessel and aspirated into the aspiration catheter 2835, the delivery catheter 2848 is extracted out of the vessel.

After the delivery catheter 2848 is extracted out of the vessel, the blood collection container 400a is disconnected from the blood filter 300. A transfusion line is thereafter attached to the outlet 409 of the blood collection container 400a and the blood collection container 400a is mounted on an IV stand.

Thereafter, the transfusion line is disposed in a blood vessel of the subject, wherein the processed autologous blood is reinfused into the patient.

### Example I

### Evaluation of the Blood Reinfusion System's Ability to Filter Thrombosed Blood

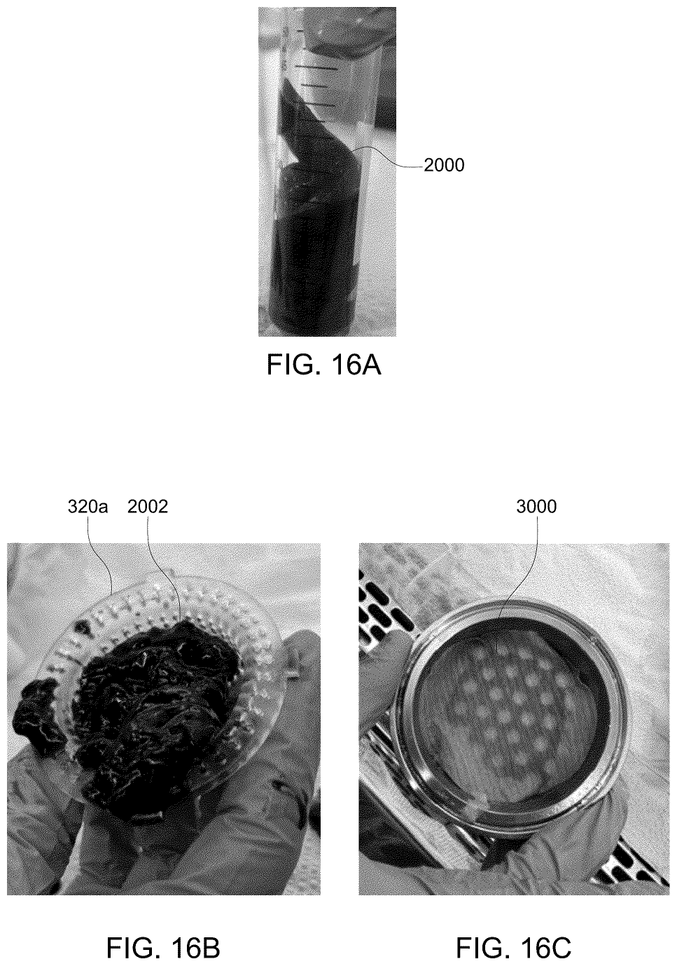

Referring now to FIG. 16A, thirty (30) cc of thrombosed bovine blood (denoted “2000”) was collected from the surgical site of a bovine animal. The thrombosed bovine blood was then combined with saline and drawn into a sixty (60) cc syringe. The 60 cc syringe containing the thrombosed blood and saline mixture was then connected to a blood delivery line in fluid communication with the blood reinfusion system 100a, depicted in FIGS. 1 and 2.

The thrombosed blood and saline mixture was then injected into and though the blood delivery line and introduced into the suction canister 200a of the blood reinfusion system 100a and into and though filters 320a, 320b, and 320c of the blood filter assembly 300.

Another 60 cc syringe was then connected to the outlet 409 of the blood collection container 400a and the processed blood was drawn into the syringe.

A ten (10) cc portion of the blood processed with the blood filter assembly 300 was then injected into a petri dish and visually examined for impurities to determine the filtration efficacy of the blood filter assembly 300 and, hence, blood reinfusion system 100a. The remaining portion of the processed blood was then filtered through a 40 μm filter (denoted 3000 in FIG. 16C) to confirm the absence of impurities.

Referring now to FIGS. 16B and 16C, there are shown the first filter 320a of the blood filter assembly 300 containing the impurities 2002 (FIG. 16B), in this instance thrombi, captured by the first filter 320a and the 40 μm filter 3000 (FIG. 16C) after the portion of the processed blood was filtered therethrough.

As depicted in FIG. 16C, the 40 μm filter 3000 was virtually devoid of impurities, and thus evidences the efficacy of the blood filter 300.

### Example II

### Evaluation of Erythrocyte Integrity and Morphology of Processed Porcine Blood

To evaluate the effect on erythrocyte integrity of porcine blood after processing with a reinfusion system of the invention, fifty (50) cc of untreated porcine blood was collected and divided into ten (10) cc and forty (40) cc samples. The 10 cc sample was left untreated and the 40 cc sample was processed via the blood reinfusion system 100a depicted in FIGS. 1 and 2 in accordance with the methods described herein.

The filtered 40 cc sample was then collected from the blood collection container 400a of the blood reinfusion system 100a for analysis.

The filtered 40 cc sample and the untreated 10 cc sample were then micro-histologically evaluated to determine erythrocyte integrity. Serum calcium (Ca), serum potassium (K), and hematocrit percent (Hct %) were determined for both the filtered 40 cc sample and the untreated 10 cc sample.

Micro-histologic evaluation of both the filtered 40 cc sample and the untreated 10 cc sample showed no significant differences in erythrocyte morphology. Based on the blood smear review, the erythrocytes and platelets in the filtered 40 cc sample and the untreated 10 cc sample similarly displayed no significant morphologic abnormalities.

As shown in Table I below, there also were no significant differences between the unfiltered control and the filtered sample in terms of serum potassium, serum calcium and Hct %.

The difference in platelet counts between the filtered 40 cc sample and the untreated 10 cc sample reflected in Table I were due to platelet clumping in the unfiltered sample.

TABLE I

| | SERUM K | SERUM Ca | | |
|---|---|---|---|---|
| | mg/dl | mg/dl | HCT % | Platelets % |
| | (Normal) | (Normal) | (Normal) | (Normal) |
| CONTROL | 4.2 | 10.0 | 33 (28-40) | 152 (200 − |
| (UNFILTERED) | (3.5-5.5) | (7.2-11) | | 800 × 1000) |
| SAMPLE | 4.3 | 10.1 | 32 (28-40) | 181 (200 − |
| (FILTERED) | (3.5-5.5) | (7.2-11.5) | | 800 × 1000) |

### Example III

### Evaluation of Erythrocyte Integrity and Morphology of Processed Human Blood

To evaluate the effect on erythrocyte integrity of human blood after processing with a reinfusion system of the invention, fifty (50) cc of untreated human blood was collected and divided into ten (10) cc and forty (40) cc samples. The 10 cc sample was left untreated and the 40 cc sample was processed via the blood reinfusion system 100a depicted in FIGS. 1 and 2 in accordance with the methods described herein.

The filtered 40 cc sample was then collected from the blood collection container 400a of the blood reinfusion system 100a for analysis.

The filtered 40 cc sample and the untreated 10 cc sample were then micro-histologically evaluated to determine erythrocyte integrity. Lactate dehydrogenase (LDH), total bilirubin, aspartate aminotransferase (AST), alanine transaminase (ALT), albumin, serum potassium (K), hematocrit percent (Hct %), and platelet concentration were similarly determined for the filtered 40 cc sample and the untreated 10 cc sample.

Micro-histologic evaluation of both the filtered 40 cc sample and the untreated 10 cc sample similarly showed no significant differences in erythrocyte morphology. The erythrocytes and platelets in the filtered 40 cc sample and the untreated 10 cc sample similarly reflected no significant morphologic abnormalities.

As shown in Table II below, there were also no significant differences between the unfiltered control and the filtered sample in terms of total bilirubin, aspartate aminotransferase (AST), alanine transaminase (ALT), albumin, serum potassium (K), hematocrit percent (Hct %), and platelet concentration.

TABLE II

| | CONTROL UNFILTERED | SAMPLE FILTERED |
|---|---|---|
| | (Normal) | (Normal) |
| LDH | 171 U/L (120-246) | 323 U/L (120-246) |
| TOTAL BILIRUBIN | 0.5 mg/dL (0.2-1.1) | 0.4 mg/dL (0.2-1.1) |
| AST | 27 U/L (0-34) | 32 U/L (0-34) |
| ALT | 43 U/L (10-49) | 44 U/L (10-49) |
| ALBUMIN | 4.9 g/dl (3.2-4.8) | 4.6 (3.2-4.8) |
| K | 4.7 mmol/L (3.5-5.1) | 4.6 mmol/L (3.5-5.1) |
| HCT | 49.4% (40-51) | 47% (40-51) |
| PLATELET | 286 K/μL (150-400) | 285 K/μL (150-400) |
| BLOOD SMEAR | Normal | Normal |

Thus, as will readily be appreciated by one having ordinary skill in the art, the present invention provides numerous significant advantages compared to prior art blood reinfusion systems and methods. Among the advantages are the following:
the provision of improved blood reinfusion systems, apparatus and methods adapted to process autologous blood with minimal, if any, effect on the quality of the blood;
the provision of improved blood reinfusion systems, apparatus and methods adapted to process autologous blood with minimal, if any, effect on total bilirubin, aspartate aminotransferase (AST), alanine transaminase (ALT), albumin, serum potassium (K), hematocrit percent (Hct %), and platelet concentration;
the provision of improved blood reinfusion systems, apparatus and methods adapted to process autologous blood with minimal blood component loss; specifically, platelet, white blood cell, plasma protein, and antibody, loss;
the provision of improved blood reinfusion systems, apparatus and methods that can be employed in sterile environments;
the provision of improved blood reinfusion systems and apparatus that are simple to use and can be easily operated manually by a single operator;
the provision of improved blood reinfusion systems and apparatus that can be promptly employed in emergency situations; and
the provision of improved blood reinfusion systems, apparatus and methods that can be employed in a multitude of surgical and interventional medical procedures.

Without departing from the spirit and scope of this invention, one of ordinary skill in the art can make various changes and modifications to the invention to adapt it to various usages and conditions. As such, these changes and modifications are properly, equitably, and intended to be, within the full range of equivalence of the following claims.

1. A method for processing autologous blood for reinfusion into a patient, comprising the steps of:
providing a blood reinfusion system, said blood reinfusion system comprising a suction canister, a blood filter assembly and a blood collection container,
said suction canister configured and adapted to receive said autologous blood therein, said suction canister comprising first filter means for extracting first impurities from said autologous blood, whereby first processed autologous blood is obtained,
said blood filter assembly configured and adapted to receive said first processed autologous blood from said suction canister, said blood filter assembly comprising second filter means for extracting excess impurities from said first processed autologous blood, whereby purified autologous blood is obtained,
said blood collection container configured and adapted to receive said purified autologous blood from said blood filter assembly; and
delivering first autologous blood into said blood reinfusion system, wherein first purified autologous blood is obtained and collected in said blood collection container.

2. The method of claim 1, wherein said first processed autologous blood said flows through said second filter means of said filter assembly solely by a gravitational force.

3. The method of claim 1, wherein said first filter means comprises a first filter comprising a pore size in the range of 1.0 mm to 5.0 mm.

4. The method of claim 1, wherein said second filter means comprises a plurality of filters.

5. The method of claim 4, wherein said plurality of filters comprises a second filter comprising a pore size in the range of 1.0 mm to 5.0 mm.

6. The method of claim 4, wherein said plurality of filters comprises a third filter comprising a pore size in the range of 40.0 micron to 1000.0 micron.

7. The method of claim 4, wherein said plurality of filters comprises a fourth filter comprising a pore size in the range of 10.0 micron to 40.0 micron.

8. The method of claim 1, wherein said blood filter assembly is detachably coupled to said suction canister.

9. A method for processing autologous blood for reinfusion into a patient, comprising the steps of:
providing means for extracting impurities from said autologous blood, said means for extracting impurities from said autologous blood comprising first filter means for extracting first impurities from said autologous blood, whereby first processed autologous blood is obtained, and second filter means for extracting excess impurities from said first processed autologous blood, whereby purified autologous blood is obtained; and
delivering first autologous blood into said means for extracting impurities from said autologous blood, wherein said first autologous blood flows into and through said first filter means and said second filter means, whereby first purified autologous blood is obtained.

10. The method of claim 9, wherein said first autologous blood flows into and through said second filter means solely by a gravitational force.

11. The method of claim 9, wherein said first filter means is detachably coupled to said second filter means.

12. The method of claim 9, wherein said first filter means comprises a first filter comprising a pore size in the range of 1.0 mm to 5.0 mm.

13. The method of claim 9, wherein said second filter means comprises a plurality of filters.

14. The method of claim 13, wherein said plurality of filters comprises a second filter and a third filter.

15. The method of claim 14, wherein said second filter comprises a pore size in the range of 1.0 mm to 5.0 mm.

16. The method of claim 14, wherein said third filter comprises a pore size in the range of 40.0 micron to 1000.0 micron.

17. The method of claim 13, wherein said plurality of filters comprises a fourth filter comprising a pore size in the range of 10.0 micron to 40.0 micron.

18. The method of claim 9, wherein said first autologous blood comprises autologous blood from an incision site of a patient.
